(19) United States
(12) Patent Application Publication
(10) Pub. No.: US 2025/0367011 A1
(43) Pub. Date: Dec. 4, 2025

(54) ENDOVASCULAR IMPLANT COUPLING AND RELEASE MECHANISM

(71) Applicant: Mark Dias, Sparks, NV (US)

(72) Inventor: Mark Dias, Sparks, NV (US)

(21) Appl. No.: 19/179,448

(22) Filed: Apr. 15, 2025

Related U.S. Application Data
(60) Provisional application No. 63/635,294, filed on Apr. 17, 2024.

(51) Int. Cl. A61F 2/95; A61B 17/00; A61B 17/12

(52) U.S. Cl. CPC A61F 2/95; A61B 17/1214; A61B 2017/00477; A61B 2017/00862; A61B 2017/1205; A61F 2002/9505

(57) ABSTRACT

An implant manipulation system includes a delivery tube, a collet, and an actuator. The collet has a plurality of flexible fingers. The actuator is configured to extend through the delivery tube and the collet and to displace the plurality of fingers radially outward into an expanded configuration. The collet has an outer diameter in a resting configuration and an outer diameter in the expanded configuration that is greater than the outer diameter in the resting configuration.

### FIELD

The present disclosure is directed to endovascular implants, such as embolic coils and stents, and particularly to implant manipulation systems associated with installation and/or removal of such implants as well as methods of use thereof.

### BACKGROUND

Endovascular treatment of medical conditions may include placement of an implant within a blood vessel. An implant may be installed permanently or may be retrieved after a period of time has elapsed. One such treatment utilizes an embolizing coil to occlude blood flow at a treatment site, thereby physically impeding blood flow and/or promoting thrombus formation. Such treatments may be beneficial when it is desired to reduce vascularization, for example when treating an aneurysm or tumor. Another such treatment utilizes a stent to widen and/or hold open a blood vessel and improve blood flow, particularly where narrowing of the blood vessel has occurred.

Implants are typically placed at a treatment site within the vasculature using a catheter to access the treatment site and a pusher to deploy the implant. A flexible small diameter catheter may be navigated to the treatment site by a guidewire or flow-directed means. Once the catheter is positioned at or near the treatment site, the implant may be inserted into the proximal end of the catheter and advanced with a pusher to the distal end of the catheter. An implant may be inserted into the catheter prior to navigating the catheter to the treatment site such that the pusher and catheter are navigated through the vasculature after assembly. Pushers have a distal end configured to engage the implant and to push the implant through the working channel of the catheter as the pusher is advanced. Once the implant reaches the distal end of the catheter, it is expelled from the distal end of the catheter by the pusher, placed at the treatment site, and released by the pusher.

A variety of coupling mechanisms have been developed with the intent to retain a coil in engagement with the pusher during delivery yet effectively release the coil when it is positioned at the treatment site. One such technique includes bonding the implant to the distal end of the pusher at a joint between dissimilar metals of the pusher and implant. After the implant is positioned at the treatment site, a small electrical current is routed through the pusher. The current severs the joint via electrolysis. The electric current may aid in thrombus formation at the treatment site. However, electrolytic release of the implant requires application of the electric current for a period time, preventing rapid detachment of the implant and potentially extending operation time.

Other techniques for coupling and subsequent detachment of an implant from a pusher utilize a mechanical connection between the implant and the pusher. For example, a proximal end of an implant may be fitted with a protrusion and the distal end of a pusher may be fitted with a collar. The collar may have an outer diameter that exceeds an inner diameter of the delivery catheter such that the internal wall of the catheter compresses the collar around the protrusion, thereby gripping the implant. When the pusher is advanced from the distal end of the catheter, the collar expands and releases the implant. One such example can be seen in U.S. Pat. No. 9,155,649. However, friction between the collar and the catheter can cause binding, preventing controlled advancement of the pusher within the catheter. Additionally, if the pusher is unintentionally advanced such that the collar protrudes from the distal end of the catheter, the implant may be prematurely deployed.

In another example, the pusher may include a narrowed internal cross-section near its distal end that retains a protrusion on the implant. Once the implant is positioned at the treatment site, a push-rod may be advanced through the pusher to force the protrusion out of the distal end of the pusher. One such example can be seen in U.S. pat. No. 5,350,397. However, due to the tortuous path of the catheter and pusher navigating through vasculature to a treatment site, the push-rod must have some degree of flexibility. The push-rod may have insufficient rigidity to force the protrusion out of the narrowed cross-section of the pusher, resulting in a failure to release the implant from the pusher.

Accordingly, improvements to coupling mechanisms that facilitate delivery while reducing premature or failed deployment are desirable.

### SUMMARY

Consistent with an aspect of the present disclosure, an implant manipulation system may include a delivery tube, a collet, and an actuator. The collet may include a plurality of flexible fingers. The collet may have an outer diameter in a resting configuration. The actuator may be configured to extend through the delivery tube and the collet and to displace the plurality of fingers radially outward into an expanded configuration. The collet may have an outer diameter in the expanded configuration that is greater than the outer diameter in the resting configuration.

In some examples, the actuator may include a wire and the plurality of flexible fingers may be configured to return to the resting configuration upon removal of the wire from the collet. The actuator may include a handle at a proximal end of the wire. The handle may be configured to slide relative to a hollow base portion of the delivery tube.

In some examples, the plurality of flexible fingers may comprise at least two flexible fingers.

In some examples, the collet may be partially disposed within a working channel of the delivery tube. The collet may be secured to the delivery tube by an adhesive.

In some examples, at least one flexible finger of the plurality of flexible fingers may have a flange extending radially outward from a distal end of the at least one flexible finger.

In some examples, the collet may have a central channel extending from a proximal end of the collet through the plurality of flexible fingers.

In some examples, a first flexible finger of the plurality of flexible fingers has a length that exceeds a length of a second flexible finger of the plurality of flexible fingers.

In some examples, an outer surface formed by the plurality of flexible fingers may be threaded.

In some examples, an implant manipulation system may further include a retainer. The retainer may comprise a tubular member with a cavity and an internal lip disposed at a proximal end of the cavity. The retainer may be configured to be affixed to a proximal end of an implant. A diameter of an opening formed by the internal lip may be less than the outer diameter of the collet in the expanded configuration and greater than the outer diameter of the collet in the resting configuration. The retainer may be integrally formed with an implant. The implant may comprise an embolic coil or a stent.

In some examples, an implant manipulation system may further include a retainer secured to a distal end of the delivery tube. The retainer may comprise a tubular member with a cavity and an internal lip disposed at a distal end of the cavity. The collet may be configured to be secured to a proximal end of an implant. A diameter of an opening formed by the internal lip may be less than the outer diameter of the collet in the expanded configuration and greater than the outer diameter of the collet in the resting configuration.

In some examples, an implant manipulation system may further include a retainer secured to a distal end of the delivery tube. The retainer may have a central channel configured receive legs of a stent when the stent is in a collapsed configuration. The outer diameter of the collet in the resting configuration may be less than an inner diameter of the stent in the collapsed configuration and the outer diameter of the collet in the expanded configuration may be greater than the inner diameter of the stent in the collapsed configuration. The retainer may have a slot configured to receive a portion of the leg when the collet is in the expanded configuration.

In some examples, the collet may be configured to slide longitudinally with respect to the delivery tube. The collet may have a flange positioned within a chamber in the delivery tube. The flange may have an outer diameter that is greater than an inner diameter of the delivery tube proximal of the chamber and distal of the chamber. The collet may further have a proximal stopper. The actuator may include a collar. Retracting the actuator in a proximal direction may cause the collar to engage the proximal stopper and retract the collet with respect to the delivery tube.

Consistent with an aspect of the present disclosure, an implant manipulation system may include a delivery tube, a collar, and an actuator. The collar may be secured to a distal end of the delivery tube. The collar may comprise at least one flexible finger. The actuator may be configured to extend through the delivery tube and the collar to displace the at least one flexible finger radially outward into an expanded configuration. In the expanded configuration, the at least one flexible finger and a distal tip of the actuator may diverge from a longitudinal axis of the delivery tube.

Consistent with an aspect of the present disclosure, an implant manipulation system may include a delivery tube, a collar, and an actuator. The collar may be secured to a distal end of the delivery tube. The collar may include a central longitudinal bore, a transverse bore intersecting the central longitudinal bore, and a ball positioned at least partially within the transverse bore. The actuator may be configured to extend through the delivery tube and the collar to displace the ball radially outward into an expanded configuration. In the expanded configuration, the ball may extend radially outward from an outer surface of the collar.

In some examples, an implant manipulation system may further include a flexible membrane around the collar. Displacement of the ball radially outward into the expanded configuration may expand the flexible membrane into a protruding configuration. The flexible membrane may be configured to displace the ball radially inward when the actuator is removed from the collar. An implant manipulation system may further include a retainer. The retainer may comprise a tubular member with a cavity and an internal lip disposed at a proximal end of the cavity. An outer diameter of the flexible membrane in the protruding configuration may be greater than an inner diameter of the internal lip. An implant manipulation system may further include a second ball positioned at least partially within the transverse bore. The actuator may be configured to displace the second ball radially outward.

Consistent with an aspect of the present disclosure, an implant manipulation system includes a delivery tube, a flexible collar, and an actuator. The flexible collar may be secured to a distal end of the delivery tube. The flexible collar may have central longitudinal bore having a proximal portion with a first inner diameter and a distal portion with a second inner diameter. The second inner diameter may be smaller than the first inner diameter in a resting configuration of the flexible collar. The actuator may be configured to extend through the collar and radially expand the distal portion of the flexible collar into an expanded configuration. The distal portion may have a greater outer diameter in the expanded configuration than in the resting configuration.

In some examples, the outer diameter of the distal portion in the expanded configuration may be greater than an inner diameter of an implant configured for use with the implant manipulation system.

Consistent with an aspect of the present disclosure, an implant system may include a delivery tube with a collet secured to a distal end of the delivery tube, a flexible coil implant, and a filament. The filament may have a distal end secured to a distal end of the implant and a free proximal end. The filament may be configured to be gripped between the collet and an internal wall of the implant when the implant is secured to the delivery tube by the collet.

Consistent with an aspect of the present disclosure, an implant manipulation system may include a delivery tube, an implant retainer, and an actuator. The actuator may be configured to extend through the delivery tube and to actuate the implant retainer to release an implant when a proximal end of the actuator is pulled in a proximal direction. The actuator may comprise a pin configured to engage the implant retainer to retain the implant and a flexible filament extending proximally from the pin.

In some examples, the flexible filament may extend proximally to a proximal end of the delivery tube. The actuator may further comprise a pull-wire secured to a proximal end of the flexible filament.

Consistent with an aspect of the present disclosure, an implant may include a stent or an embolic coil and a retainer secured to a proximal end of the stent or embolic coil. The retainer may include a tubular member with a cavity and an internal lip disposed at a proximal end of the cavity. The cavity may be configured to receive a retention mechanism of an implant pusher.

Consistent with an aspect of the present disclosure, an implant may include a stent or an embolic coil and a collet secured to a proximal end of the stent or embolic coil. The collet may have a plurality of flexible fingers. The collet may be configured for receipt within a retainer secured to a delivery tube and may be configured to receive a portion of an actuator between the plurality of flexible fingers to expand the collet into an expanded configuration in which the collet is interlocked with the retainer.

Other examples include corresponding methods, systems, apparatuses, and devices, each related to the functions described herein.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTION OF THE DRAWINGS

The accompanying drawings illustrate implementations of the systems, devices, and methods disclosed herein and together with the description serve to explain the principles of the present disclosure.

FIG. 1 is an exploded view of an example of an implant manipulation system in accordance with an example of the present disclosure.

FIG. 2 is a cross-sectional view of the implant manipulation system of FIG. 1 with an implant.

FIGS. 3A-3E illustrate an example of a collet of a retention mechanism in accordance with the present disclosure.

FIG. 4 illustrates the implant manipulation system of FIG. 1 in a deployed configuration.

FIGS. 5A-5B illustrate an example of a release mechanism in accordance with the present disclosure.

FIG. 6 illustrates release of an implant when the release mechanism of FIGS. 5A-5B is operated.

FIG. 7 illustrates an example of a collet of a retention mechanism in accordance with the present disclosure.

FIGS. 8A-8B illustrate an example of a collet of a retention mechanism in accordance with the present disclosure.

FIGS. 9A-9C illustrate an example of a collet of a retention mechanism in accordance with the present disclosure.

FIGS. 10A-10E illustrate an example of an implant manipulation system with an implant having a retainer in accordance with the present disclosure.

FIGS. 10F-10G illustrate an example of an implant manipulation system with a delivery tube having a retainer in accordance with the present disclosure.

FIGS. 11A-11E illustrate an example of a retention mechanism in accordance with the present disclosure.

FIGS. 12A-12E illustrate an example of a retention mechanism with a movable collet in accordance with the present disclosure.

FIG. 13 illustrates an example of a collar of a retention mechanism in accordance with the present disclosure.

FIGS. 14A-14E illustrate an example of a retention mechanism in accordance with the present disclosure.

FIGS. 15A-15B illustrate an example of a retention mechanism in accordance with the present disclosure.

FIG. 16 illustrates an example of an implant with a stretch-resistant element in accordance with the present disclosure.

FIG. 17 illustrates an example of an actuator with a foreshortening element in accordance with the present disclosure.

Examples of the present disclosure and their advantages are best understood by referring to the detailed description that follows. It should be appreciated that like reference numerals are used to identify like elements illustrated in one or more of the figures, wherein showings therein are for purposes of illustrating examples of the present disclosure and not for purposes of limiting the same.

### DETAILED DESCRIPTION

The devices and techniques disclosed in this document may be used to place and/or retrieve medical implants, particularly endovascular implants such as embolic coils and stents.

In the following description, like elements are marked throughout the specification and drawings with similar reference numerals. The drawing figures are not necessarily drawn to scale and certain elements are shown in generalized or schematic form in the interest of clarity and conciseness. It should be understood that the embodiments of the disclosure herein described are merely illustrative of the principles of the invention.

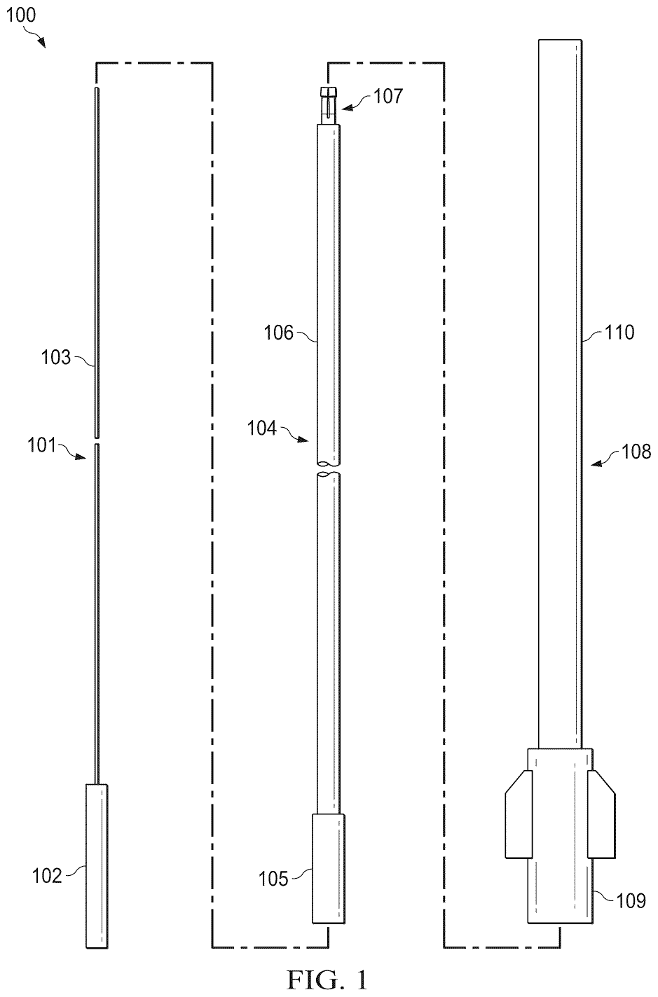

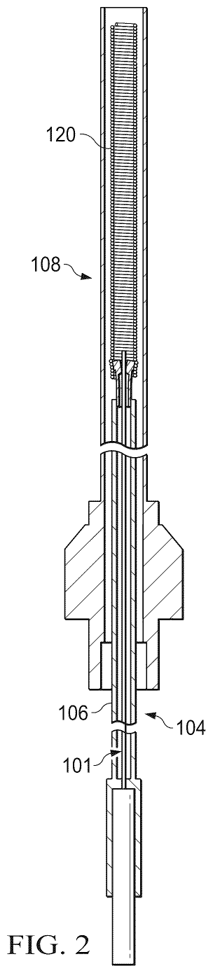

FIGS. 1 and 2 illustrate an example of an implant manipulation system 100. The implant manipulation system 100 may be used to navigate to a treatment site within a patient anatomy and place an implant at the treatment site. The implant manipulation system 100 includes an actuator 101 and a delivery tube 104, which collectively form a “pusher.” The actuator 101 includes a handle 102 and a wire 103 extending from the handle 102. The wire 103 may be a single strand, a woven cable, or the like and is flexible or semi-rigid. The delivery tube 104 includes a base 105, a shaft 106, and a retention mechanism at a distal end of the shaft. The delivery tube may be constructed from any suitable material(s), including but not limit to stainless steel, a stainless steel alloy, nickel-titanium, a nickel-titanium alloy, or a polymer. The delivery tube 104 may have an outer diameter in a range of 0.010″-0.020″ and an inner diameter of the working channel in a range of 0.0050″-0.016″. A length of the delivery tube may be in a range of 60″-80″. These dimensions are exemplary only and are not intended to limit the scope of the present disclosure. It should be appreciated that the actual dimensions may vary between 10%-200% of these example dimensions without departing from the scope of the disclosure. In the illustrated example, the retention mechanism comprises a collet 107, discussed in further detail below. The retention mechanism is configured to retain an implant 120, such as a nitinol embolic coil, in engagement with the pusher during navigation to the treatment site and to selectively release the implant 120 at the treatment site by operation of the actuator 101. The actuator 101 is configured to be positioned within the delivery tube 104 with the handle 102 disposed in the base 105 and the wire 103 extending through the shaft 106 and retention mechanism, including the collet 107 in the illustrated example. Optionally, the implant manipulation system 100 includes a catheter 108 that includes a hub 109 configured to remain external to the patient anatomy and a shaft 110 configured to extend into the patient anatomy to the treatment site or adjacent to the treatment site.

An operator, such as a surgeon, may navigate the catheter 108 to a position near the treatment site within the patient anatomy. In some examples, the catheter 108 may be navigated using a guidewire that has been routed to the treatment site, the guidewire being removed from the catheter 108 after the catheter is positioned. The operator may then insert the implant and pusher, including the actuator 101 and delivery tube 104, through the catheter 108. In some examples, the implant and pusher may be positioned within the catheter 108 prior to navigating the catheter to the treatment site, as shown in FIG. 2.

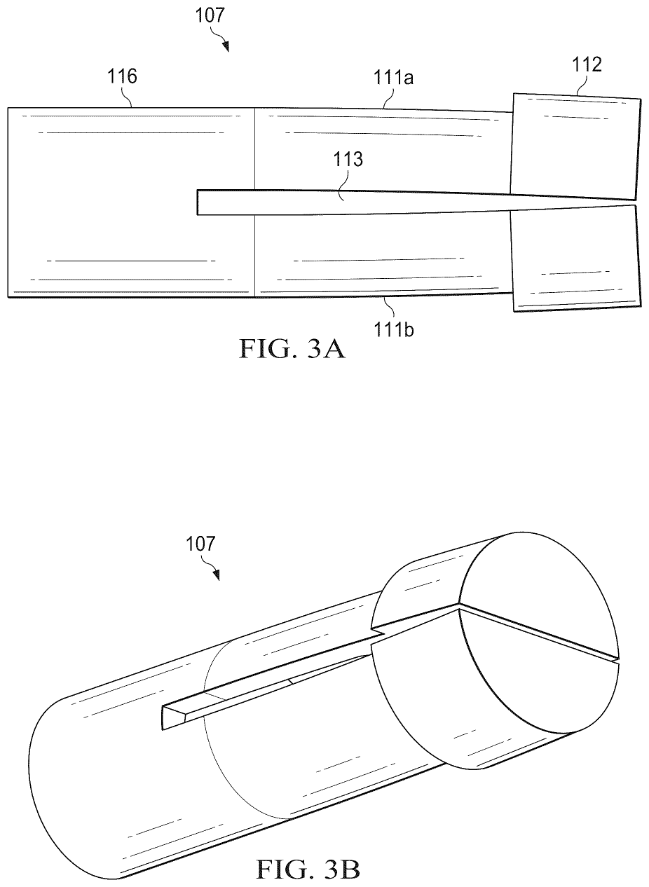

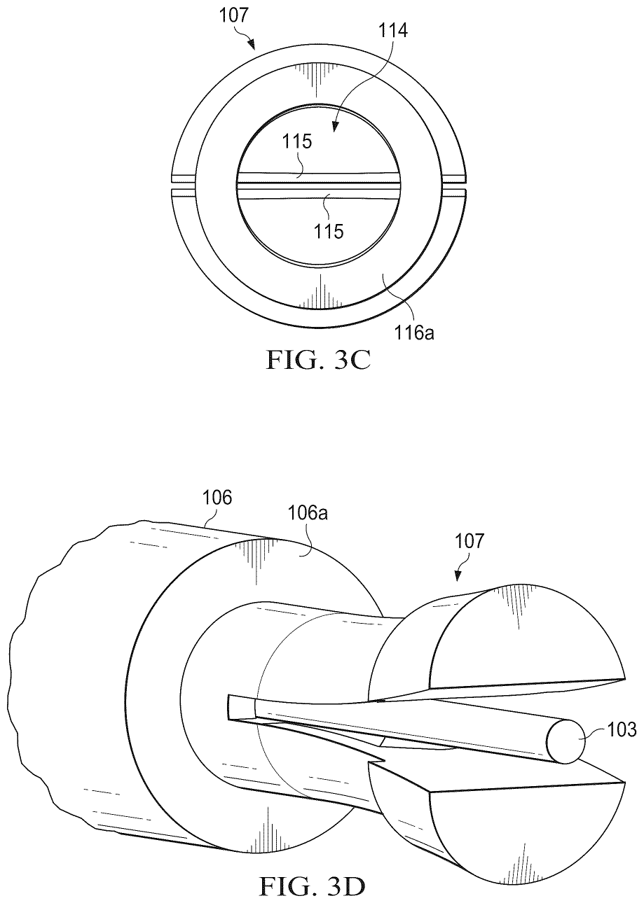

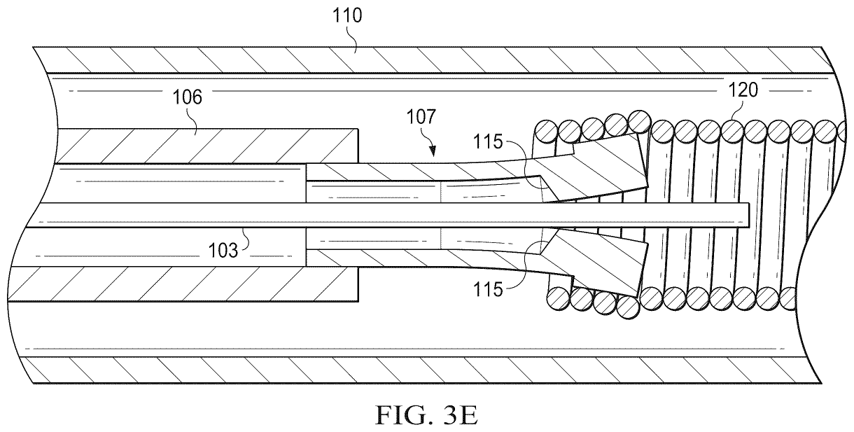

FIGS. 3A-3E illustrate an example of a collet 107 of a retention mechanism configured to retain the implant 120 in engagement with the pusher and to selectively release the implant at the treatment site. The collet 107 includes a base 116 having a central channel 114 extending through a proximal surface 116a of the base 116. The collet 107 may be secured to the delivery tube 104 by inserting at least a portion of the base 116 into the working channel of the delivery tube 104 as shown in FIG. 3E. Alternatively, the proximal surface 116a may be joined to a distal surface 106a (shown in FIG. 3D) of the shaft 106 of the delivery tube 104 to form a butt-joint. A plurality of flexible fingers 111 extend distally from the base 116. The base 116 have a diameter in a range of 0.006″-. 010″ and a length in a range of 0.015″-0.030″. The flexible fingers may have a length in a range of 0.005″-0.010″. Again, these dimensions are provided merely as an example. In the illustrated example, the collet 107 has two flexible fingers 111a, 111b. However, it will be appreciated that any suitable number of flexible fingers may be used, such as three to twelve flexible fingers disposed radially around a longitudinal axis of the collet 107. An optional flange 112 an outer surface at the distal end of the flexible fingers 111. A gap 113 is formed between the plurality of flexible fingers 111 which is in fluid communication with the central channel 114. The collet 107 is illustrated in FIGS. 3A-3C in a resting configuration in which the flexible fingers 111 are in a neutral unstressed position. The wire 103 of the actuator 101 may be routed through the central channel 114 of the collet 107 and into the gap 113 between the flexible fingers 111. One or more tapered edges 115 may optionally be formed within the gap 113 to direct the wire 103 through the gap 113 and between the flexible fingers 111. This forces the flexible fingers 111 apart into an expanded configuration as shown in FIG. 3D. The collet 107 may be configured such that an outer diameter of the collet 107 at a distal end in the resting configuration is less than an inner diameter of an implant 120 while the outer diameter of the collet 107 in the expanded configuration exceeds the inner diameter of an implant 120. For example, the outermost diameter of the collet in the resting configuration may be in a range of 0.007″-0.010″ and an outermost diameter of the collet in the expanded configuration may be in a range of 0.009″-0.012″. In this manner the flexible fingers 111, optionally including flange 112, may grip an inner surface of the implant 120. Although the implant 120 is illustrated as an embolic coil, it should be appreciated that the retention mechanism including collet 107 is contemplated for use with any suitable implant.

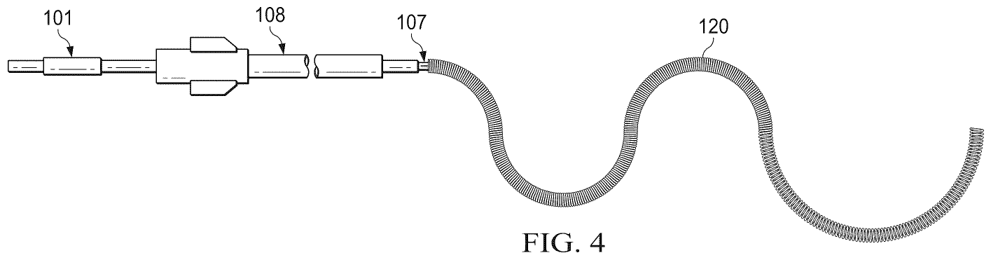

With the implant 120 secured to the pusher, the pusher positioned in the catheter 108 as shown in FIG. 2, and the catheter 108 positioned at the treatment site, the pusher (including actuator 101 and delivery tube 104) may be advanced relative to the catheter 108 to deploy the implant. For example, the operator may grip the handle 102 of the actuator 101 and/or the base 105 of the delivery tube 104 and slide the base 105 of the delivery tube 104 toward the hub 109 of the catheter 108. This relative movement between the delivery tube 104 and the catheter 108 expels the implant 120 from the distal end of the catheter 108 into a deployed configuration as shown in FIG. 4.

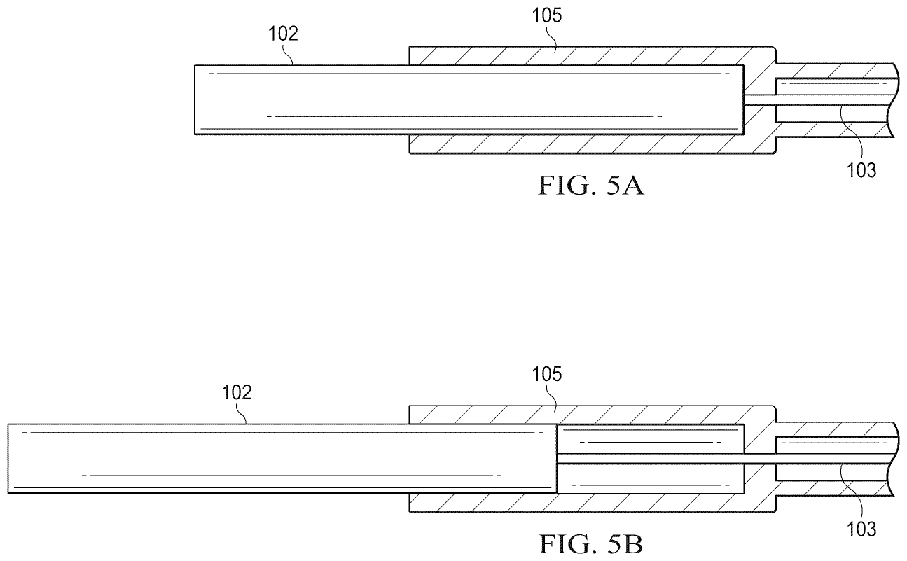

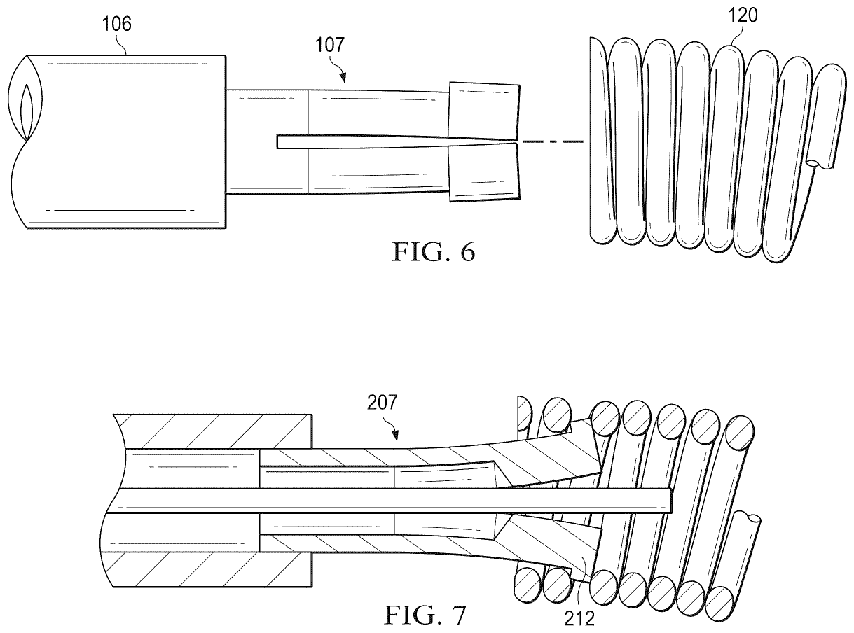

Once the implant 120 is positioned as desired by the operator, the operator can acuate a release mechanism to detach the implant 120 from the pusher. FIGS. 5A-5B illustrate an example of a release mechanism. In the configuration shown in FIG. 5A, the handle 102 of the actuator 101 is in a forward position relative to the base 105 of the delivery tube 104. In this position, the wire 103 extends through the collet 107 as shown in FIG. 3E. To detach the implant 120, the operator pulls handle 102 relative to the base 105 to slide the wire 103 proximally with respect to the delivery tube 104. This movement retracts the distal end of the wire 103 from the collet 107, allowing the flexible fingers 111 to return to the resting configuration, thereby releasing the implant 120 as shown in FIG. 6.

FIG. 7 illustrates an example of a retention mechanism of a pusher. In this example, the collet 107 is replaced by collet 207. Collet 207 is substantially similar to collet 107 and functions in a similar manner. However, collet 207 includes flexible fingers having a flange 212 that is narrower (in a longitudinal direction) than the flange 112 of collet 107. The smaller size of the flange 212 permits at least a portion of the flange 212 to become interposed between adjacent loops of an embolic coil when the collet 207 is transitioned to the expanded configuration by the actuator 101.

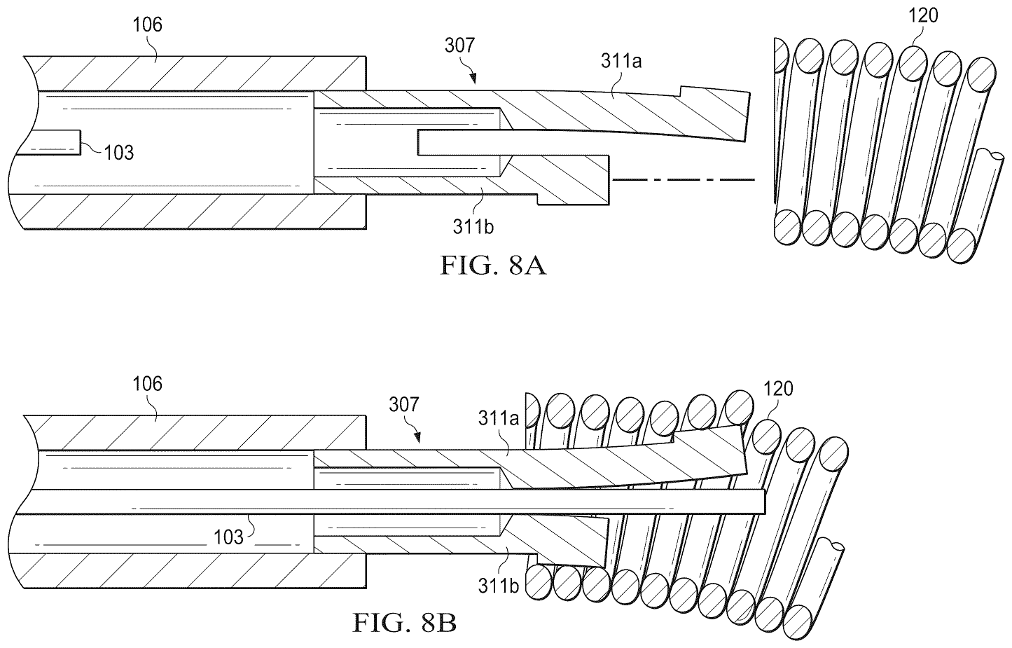

FIGS. 8A-8B illustrate another example of a retention mechanism. In this example, the collet 107 is replaced by collet 307. Collet 307 is substantially similar to collet 107 and functions in a similar manner. However, collet 307 includes flexible fingers having different lengths. Specifically, a first flexible finger 311a is longer than a second flexible finger 311b. When the collet 307 is transitioned to the expanded configuration by the actuator 101, the collet engages the implant 120 along a longer length of the coil, as compared to collet 107, which may aid in distributing the retention force over a greater area and reducing stress concentration. The flexible fingers of collet 307 may also permit use of a common diameter of wire because the longer finger(s) deflect with less resistance than shorter fingers.

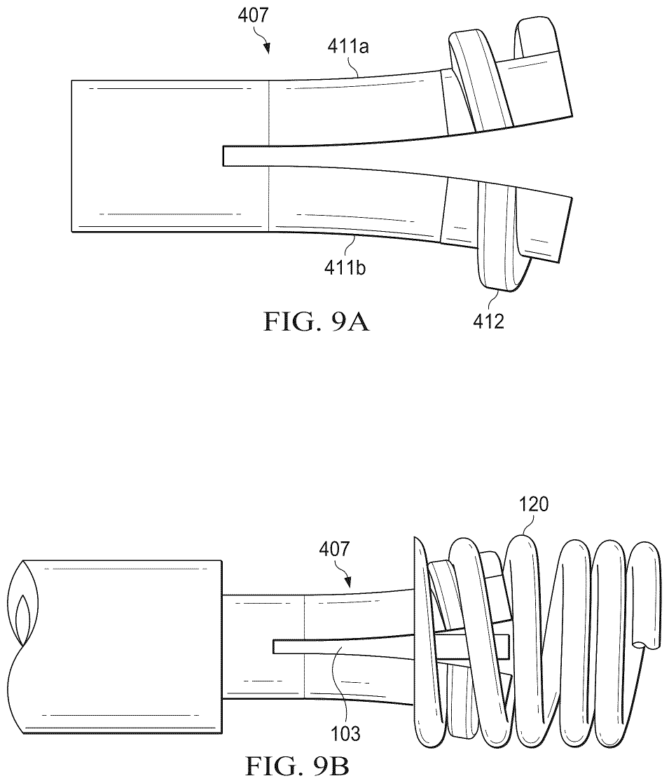

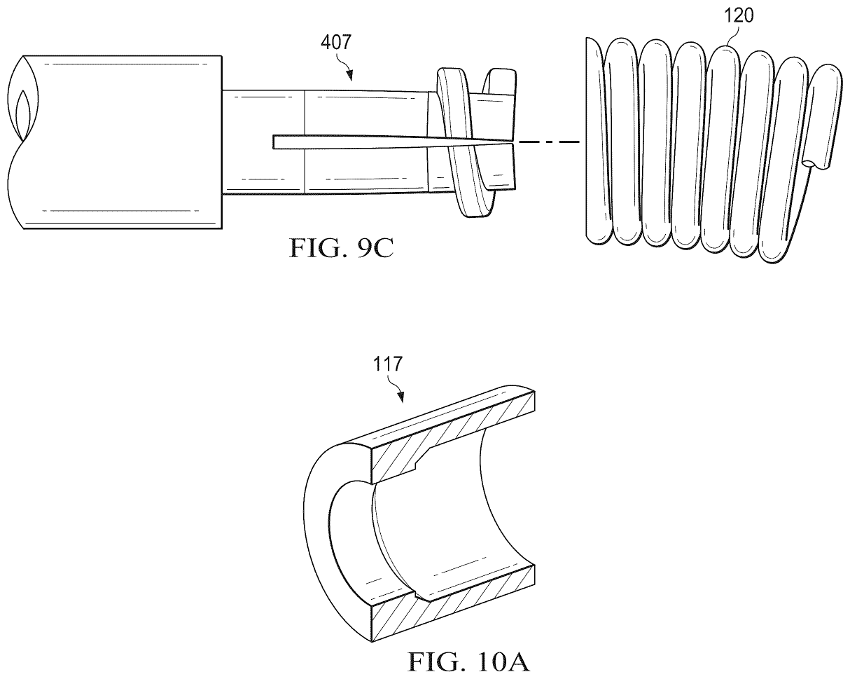

FIGS. 9A-9C illustrate yet another example of a retention mechanism. In this example, the collet 107 is replaced by collet 407. Collet 407 is substantially similar to collet 107 and functions in a similar manner. However, collet 407 includes flexible fingers 411a, 411b which are threaded around an external surface formed by the plurality of flexible fingers. A pitch of the thread 412 may be configured to match a pitch of the coil of implant 120. This may permit the thread 412 to become interposed between adjacent loops of the implant as shown in FIG. 9B which, in turn, may increase the tension force required to separate the implant 120 from the collet 407 when the collet 407 is in the expanded configuration. When the wire 103 is removed from the collet 407 and the flexible fingers 411 return to the resting configuration, the outer diameter of the thread 412 is smaller than the inner diameter of the implant 120, allowing the implant 120 to detach from the collet 407 as shown in FIG. 9C.

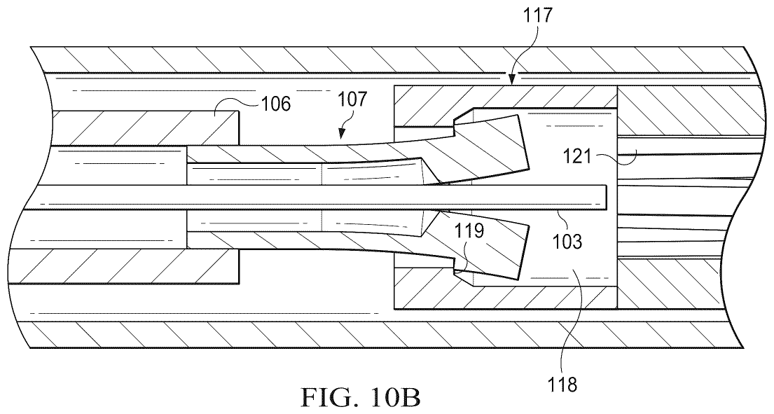

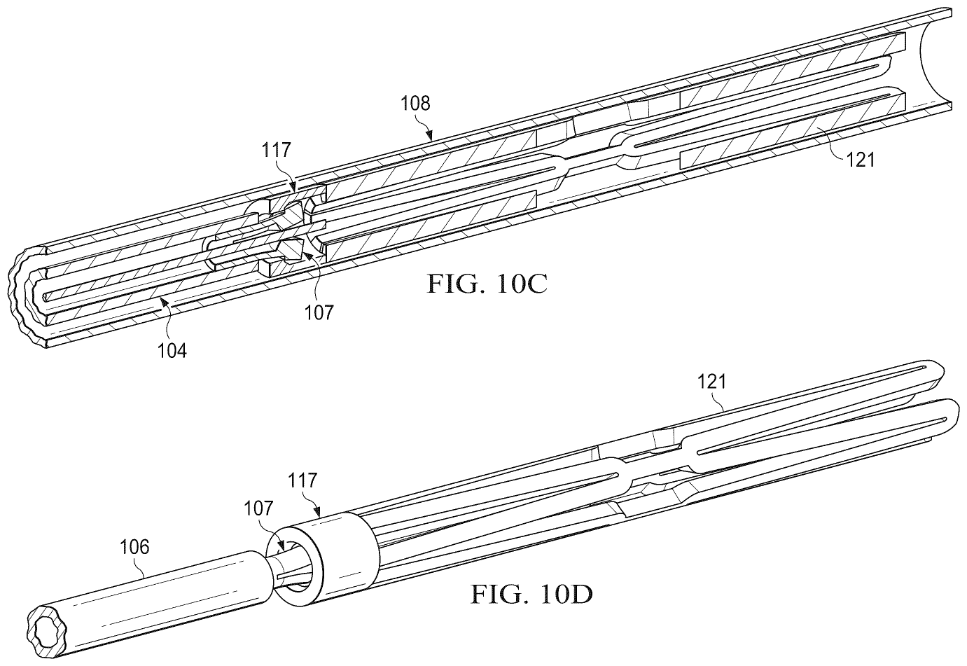

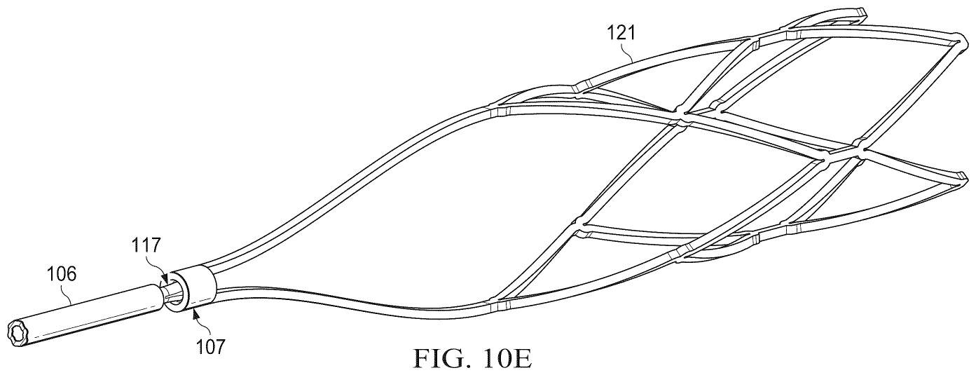

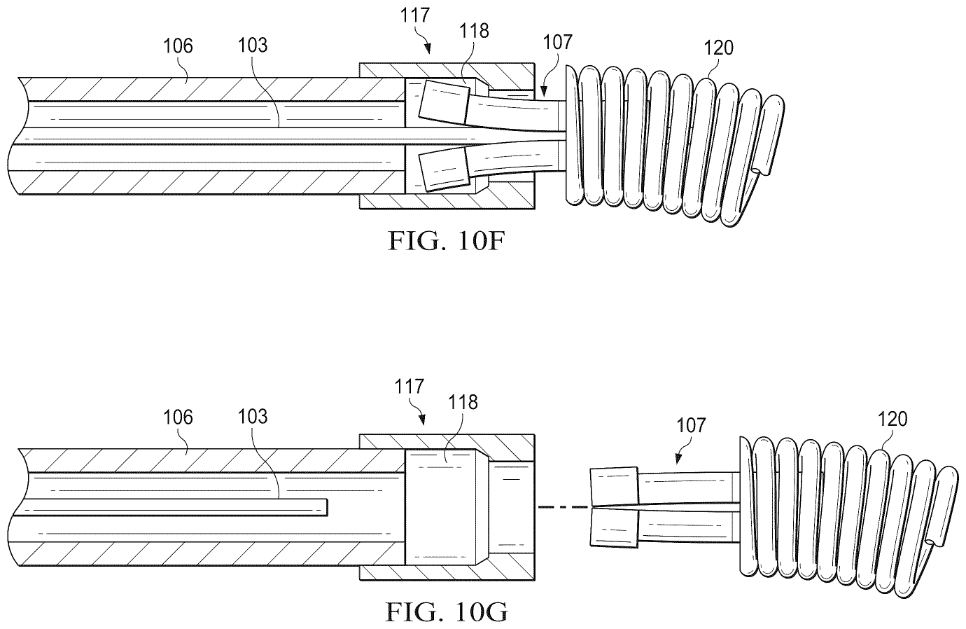

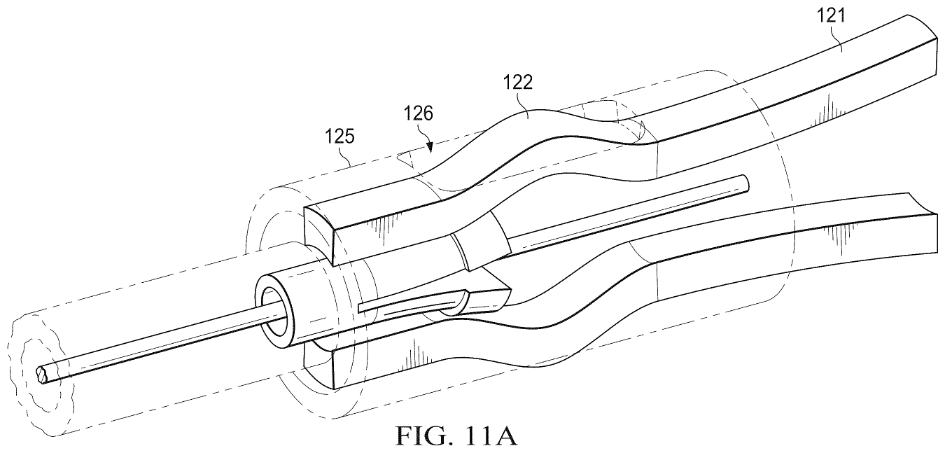

FIGS. 10A-10E illustrate another example of a retention mechanism. This retention mechanism is substantially similar to that of FIGS. 3A-3E but additionally includes a retainer 117 secured to an implant 121, illustrated as a stent. The stent may be formed of any suitable material such as nitinol or steel and may be laser-cut or braided. The retainer 117 may be laser-welded or otherwise affixed to the implant 121. Retainer 117 is formed of a substantially tubular member having an internal cavity 118 with an inner lip 119 that projects radially inward from an inner surface of the tubular member. The retention mechanism of this example functions in a substantially similar manner as the example described with reference to FIGS. 1-6, with the exception that the flexible fingers 111 of the collet 107 are received within the cavity 118 of the retainer 117 and engage the retainer 117 rather than engaging the implant directly. Due to the relative diameters of the collet 107 and inner lip 119, the inner lip 119 prevents the collet 107 from sliding out of the retainer 117 when the collet 107 is in the expanded configuration but permits the collet 107 to detach from the retainer 117 when in the resting configuration after the actuator has been removed from the collet 107. In this example, the implant 121 may be positioned within the catheter 108 in a collapsed configuration as shown in FIG. 10C and also in FIG. 10D with the catheter not shown. Advancing the delivery tube 104 and implant 121 with respect to catheter 108 in a similar manner as described above in relation to FIG. 4 expels the implant 121 from the catheter 108, allowing the implant 121 to expand into a deployed configuration as shown in FIG. 10E. The actuator 101 can then be operated to release the implant 121 from the collet 107 at the treatment site. Although illustrated and described in the context of a stent, it should be appreciated that implant 121 could be substituted with any suitable implant such as an embolic coil.

FIGS. 10F-10G illustrate another example of a retention mechanism. This retention mechanism is substantially similar to that of FIGS. 10A-10E but swaps the positioning of the collet 107 and retainer 117. More specifically, the collet 107 is reversed and secured to implant 120 and the retainer 117 is reversed and secured to the shaft 106 of the delivery tube 104. The collet 107 may be laser-welded or otherwise affixed to the implant 120. Although illustrated and described in the context of embolic coil implant, it should be appreciated that implant 120 could be substituted with any suitable implant such as a stent.

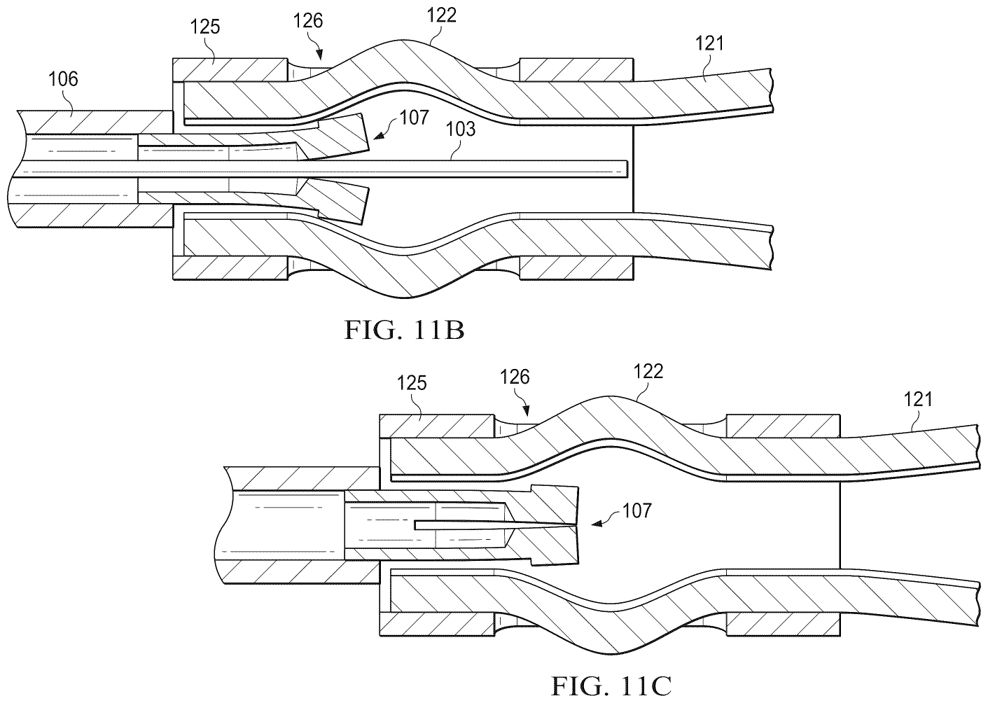

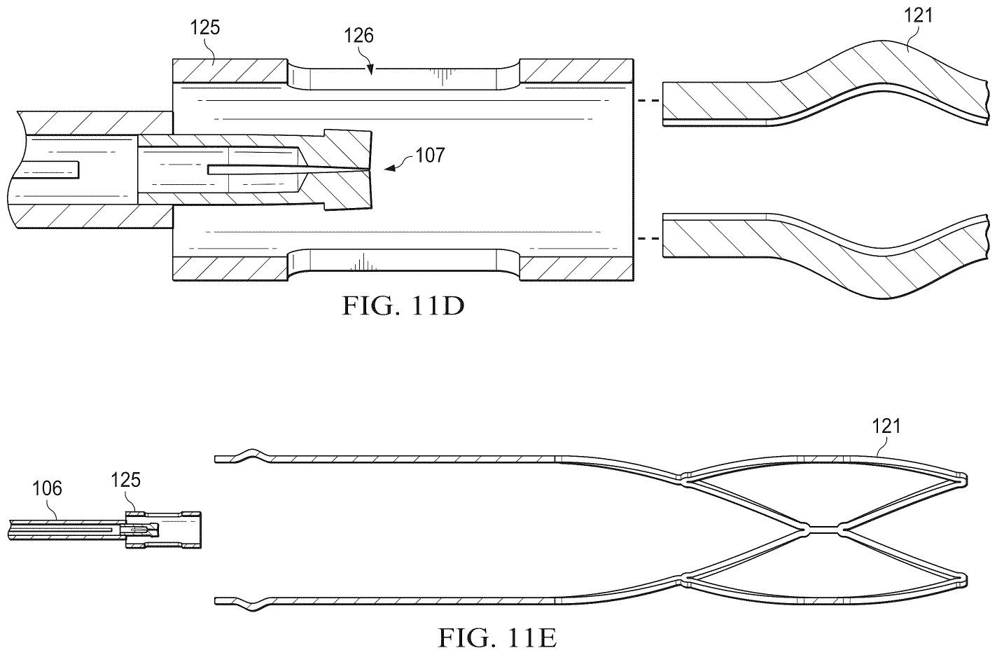

FIGS. 11A-11E illustrate another example of a retention mechanism. This retention mechanism is substantially similar to that of FIGS. 3A-3E but additionally includes a retainer 125 secured to the shaft 106 of the delivery tube 104. The retainer 125 may have a central channel configured to receive a portion of an implant. In the illustrated example, the implant 121 is a stent and legs 122 of implant 121 may be received in the retainer 125 with the implant 121 in a collapsed configuration. Optionally, the retainer 125 may include one or more openings 126 in the form of slots extending through a wall of the retainer and a protruding portion of the legs 122 may be configured for receipt within the corresponding openings 126 when the collet 107 is in the expanded configuration as shown in FIG. 11B. Upon retraction of the wire 103 of the actuator from the collet 107, the flexible fingers of the collet 107 return to their resting configuration as shown in FIG. 11C. This permits the implant 121 to detach from the retention mechanism as shown in FIG. 11D. Upon detachment, the implant 121 may spring into its deployed configuration as shown in FIG. 11E. This arrangement permits the implant 121 to fully open to achieve positive apposition against an arterial wall, for example.

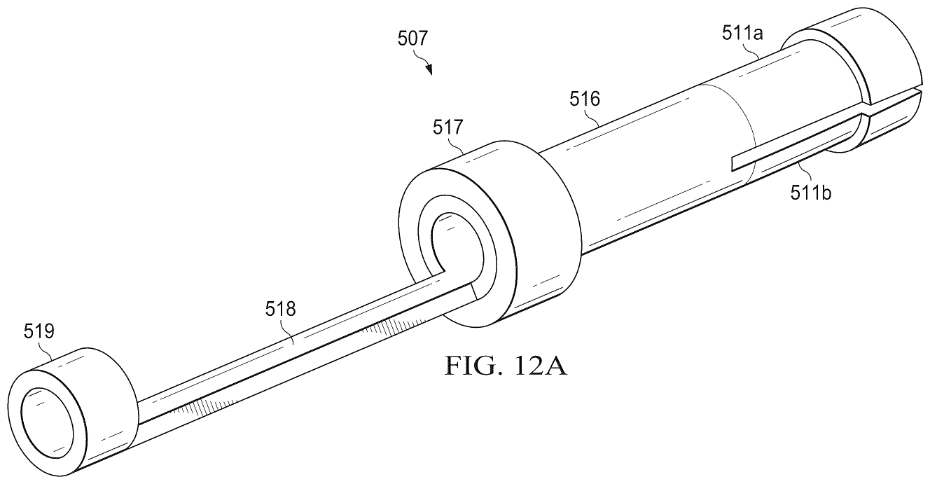

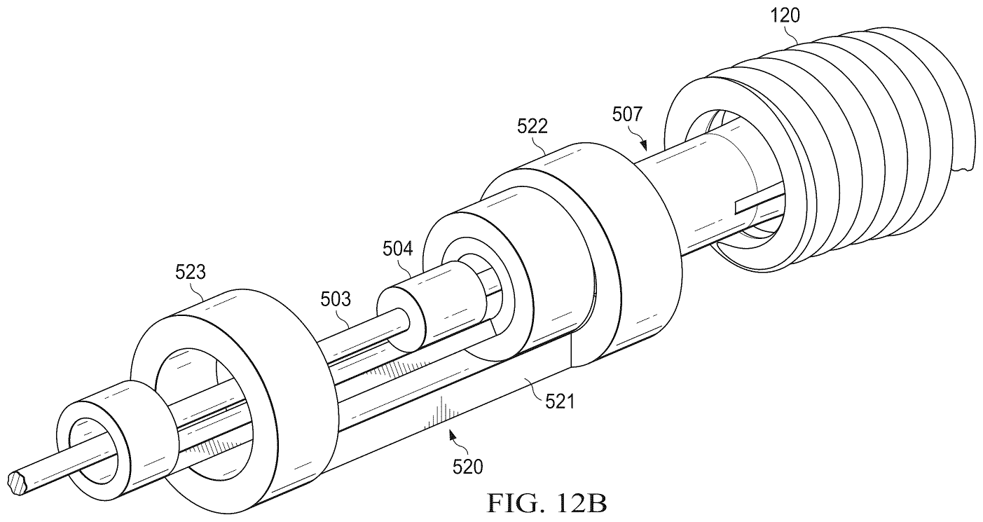

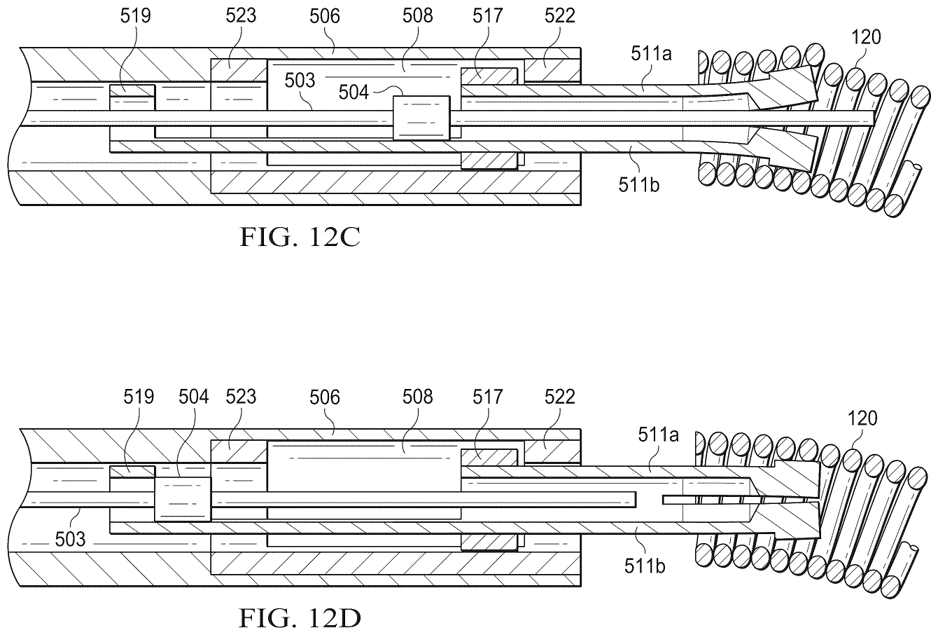

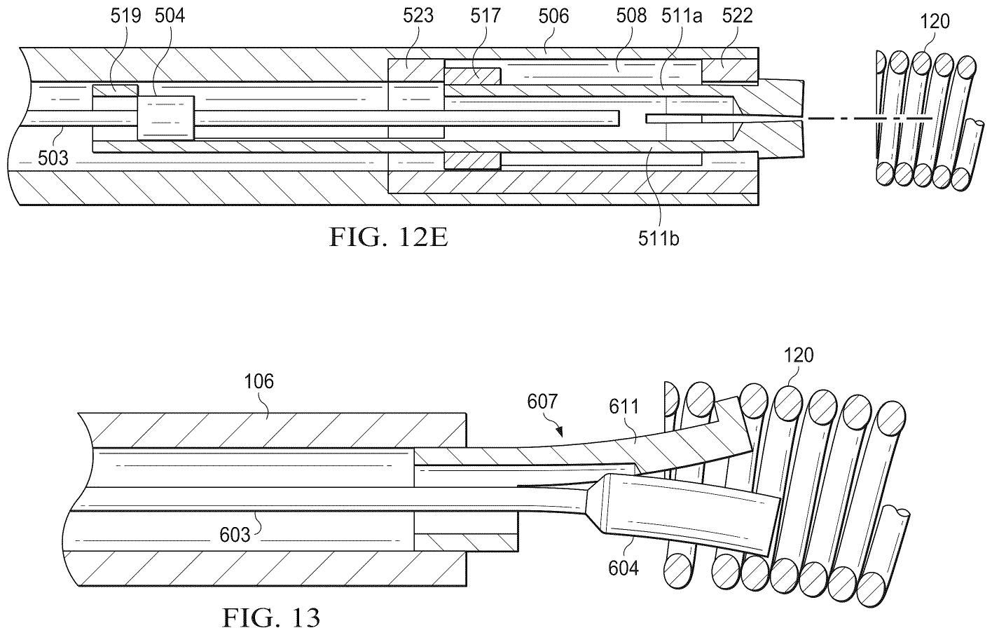

FIGS. 12A-12E illustrate another example of a retention mechanism in which collet 507 is movable with respect to a delivery tube shaft 506. Some features of collet 507 are similar to those of collet 107, such as collet 507 having a base 516 and a plurality of flexible fingers 511a, 511b. However, collet 507 further comprises a distal stopper 517 in the form of a flange at the proximal end of the base 516, a strut 518 extending proximally from the base 516, and a proximal stopper 519 at the proximal end of the strut 518 in the form of a ring. The distal stopper 517 may be formed integrally with the base 516 of collet 507 or may be an annular member affixed to the base 516. An insert 520 is secured in a distal portion of the working channel of the delivery tube shaft 506. The insert 520 includes a distal ring 522 and a proximal ring 523 secured together by a strut 521 extending between the rings. The collet 507 may be assembled with the insert 520 such that the distal stopper 517 is positioned between the distal ring 522 and proximal ring 523 of the insert 520 such that the base 516 and flexible fingers 511 project distally from the distal ring 522 as shown in FIG. 12B. The diameter of the distal stopper 517 may exceed the inner diameter of the distal ring 522 and proximal ring 523 such that the rings 522, 523 define a range of motion of the collet 507. A wire 503 of an actuator extends through the collet 507 and retains the flexible fingers 511 in engagement with implant 120. Wire 503 includes a collar 504, or other protrusion, at a fixed position along the wire 503.

FIG. 12C illustrates the assembly including the insert 520 and collet 507 of FIG. 12B positioned in a chamber 508 of the shaft 506 of a delivery tube with the collet 507 in the expanded configuration engaged with implant 120. Upon positioning of the implant 120 at the treatment site, the operator retracts the wire 503 from the flexible fingers 511, releasing the implant 120 as illustrated in FIG. 12D. Retraction of the wire 503 translates the collar 504 proximally into contact with the proximal stopper 519 of the collet 507. Continued retraction of the wire 503 causes collar 504 to pull on the proximal stopper 519, translating the collet 507 in the proximal direction. Engagement of the distal stopper 517 of the collet 507 with the proximal ring 523 of the insert 520 limits the range of motion of the collet 507 in the proximal direction as shown in FIG. 12E. If the collet 507 fails to completely release the implant 120 when the wire 503 is retracted from the flexible fingers 511, retracting the collet 507 to the position shown in FIG. 12E may pull the proximal end of the implant 120 into engagement with the distal side of the distal ring 522 of the insert 520 and/or the distal end of the shaft 506. This engagement between the implant and pusher as the collet 507 is retracted may dislodge the implant 120 from the collet 507.

FIG. 13 illustrates an example of a retention mechanism. In this example, the collet 107 is replaced by collar 607. Collar 607 is substantially similar to collet 107. However, collar 607 includes only one flexible finger 611. Wire 613 includes a cap 604 at the distal end. Advancing the wire 613 distally through the collar 607 deflects flexible finger 611 in a first direction and deflects the cap 604 in the opposite direction. The divergence of the flexible finger 611 and cap 604 causes the retention mechanism to engage the inner surface of the implant 120. Retraction of the wire 603 retracts the cap 604 into the central channel of the collar 607 and permits the flexible finger 611 to return to the resting configuration to release the implant 120.

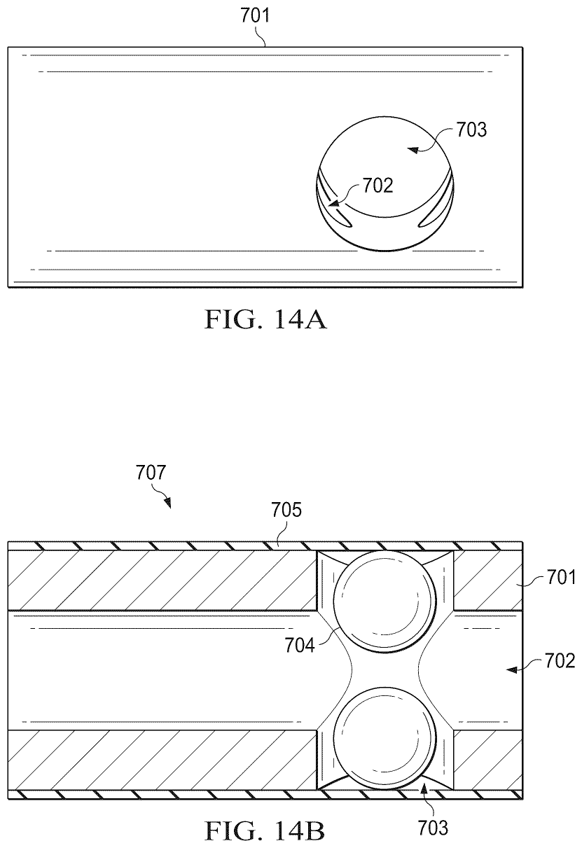

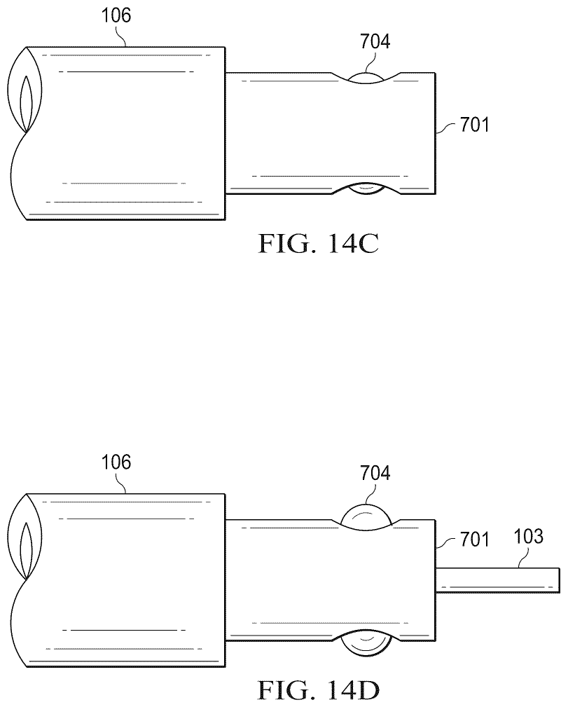

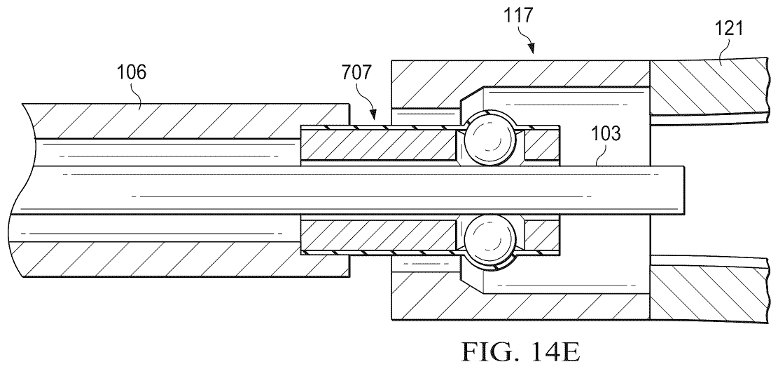

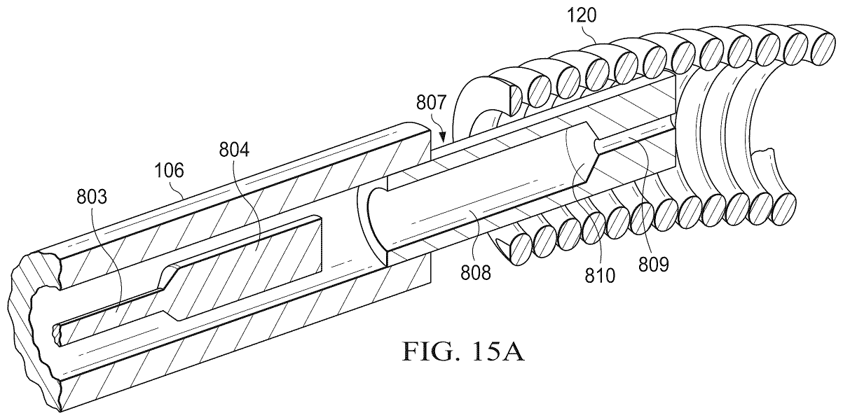

FIGS. 14A-14E illustrate an example of another retention mechanism. In this example, the collet 107 is replaced by fixture 707. Fixture 707 includes a collar 701 in the form of a cylindrical member having a central longitudinal bore 702 and a transverse bore 703 that intersects the central longitudinal bore 702. One or more balls 704, such as a ball bearing or any other suitable movable members (e.g., cylinders, cones, pins, etc.), are disposed in the transverse bore 703 and a membrane 705 surrounds at least a portion of the outer surface of the collar 701 including the radial openings of the transverse bore 703 as shown in FIG. 14B. The balls 704 may be sized such that they block one another from fully entering the central longitudinal bore 702 and instead remain positioned at least partially within the transverse bore 703. FIG. 14C illustrates the fixture secured to the shaft 106 of a deployment tube with the membrane 705 not shown to avoid obstructing the view of the balls 704. In FIG. 14C, the fixture 707 is in a resting configuration in which the membrane biases the balls 704 toward the central longitudinal bore 702. Pushing a wire 103 through the fixture 707 displaces the balls 704 radially outward in respective portions of the transverse bore 703 as shown in FIG. 14D. As shown in FIG. 14E, this causes the membrane 705 to protrude radially outward from the collar 701 to engage retainer 117 secured to implant 121. When the wire 103 is removed, the membrane 705 pushes the balls 704 radially inward back toward the central longitudinal bore 702, releasing the retainer 117. It should appreciated that the fixture 707 could also be used to engage an inner surface of an implant such as an embolic coil without the use of a retainer secured to the implant.

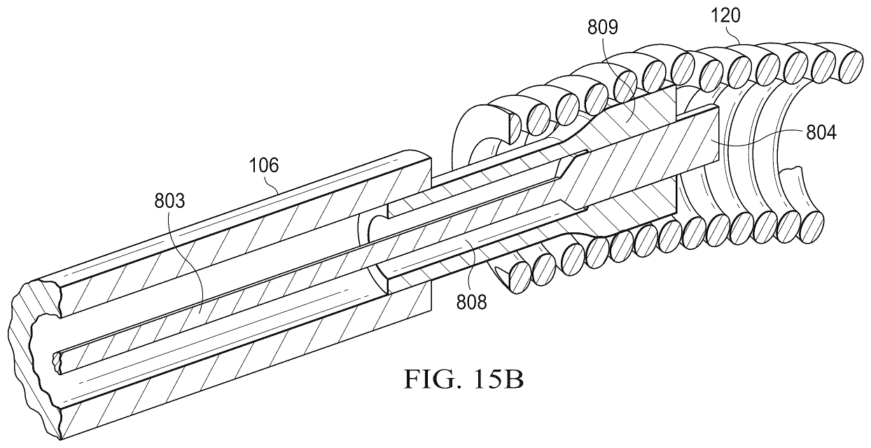

FIGS. 15A-15B illustrate another example of a retention mechanism. In this example, the collet 107 is replaced by flexible collar 807. The flexible collar 807 has a central longitudinal bore having a proximal portion 808 and a distal portion 809. The proximal portion 808 has a greater diameter in the resting configuration shown in FIG. 15A than the distal portion 809. An actuator comprising a wire 803 with a cap 804 is advanced through the shaft 106 of the delivery tube. The cap 804 has a diameter that is smaller than the diameter of the proximal portion 808 but larger than the diameter of the distal portion 809 in the resting configuration. The cap 804 engages angled surface 810 that separates the proximal portion 808 from the distal portion 809 which directs the cap 804 into the distal portion 809 as it is advanced distally. The cap 804 dilates the distal portion 809 into an expanded configuration as shown in FIG. 15B. In this configuration, the outer surface of the distal portion 809 is pressed against the inner surface of the implant 120, thereby retaining the implant 120 on the collar 807. Retraction of the wire 803 from the flexible collar 807 permits the distal portion 809 to return to its resting configuration, thereby releasing the implant 120.

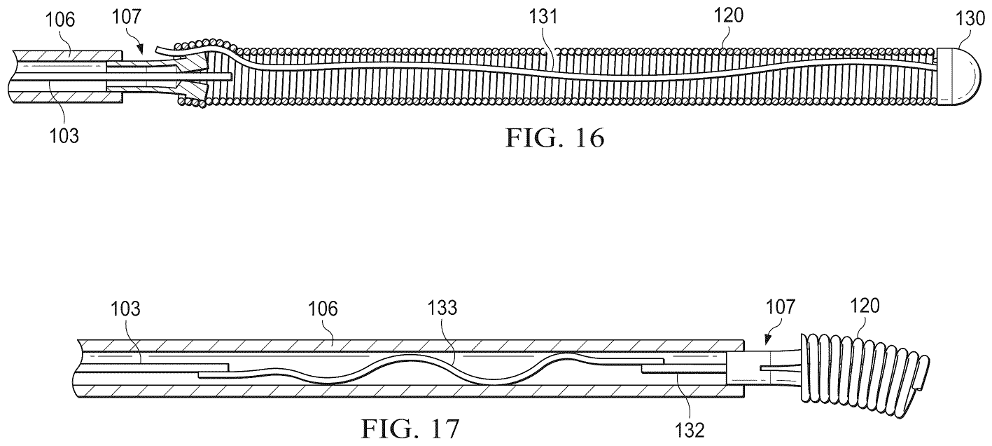

FIG. 16 illustrates an example of an implant 120 with a stretch-resistant element 131. The implant 120 in this example includes a cap 130 mounted to the distal end of the implant 120. The stretch-resistant element 131, in the form of a flexible filament although other suitable materials may be used, extends proximally through a central lumen of the implant 120. A proximal end of the flexible filament is free floating. When a user attaches the shaft 106 of the delivery tube to the implant 120 using the collet 107 and wire 103, they may pull on the free proximal end of the stretch-resistant element, optionally placing it into tension, and trap the free end between the flexible fingers of the collet 107 and the inner wall of the implant 120. The stretch-resistant element 131 so positioned may increase the implant's resistance to stretching during positioning of the implant 120 at the treatment site. The stretch-resistant element 131 may be released by retracting the wire 103, allowing the flexible fingers to return to their resting configuration, thereby effectively eliminating the resistance to stretching imparted to the implant 120 by the stretch-resistant element 131. Although shown in conjunction with the retention mechanism of FIGS. 3A-3E, it should be appreciated that the implant 120 with a stretch-resistant element 131 of FIG. 16 can be used with any other example of a retention mechanism disclosed herein.

FIG. 17 illustrates an example of an actuator with a foreshortening element 133. The actuator of FIG. 17 may be substantially similar to the actuator of FIGS. 1-3E, for example. However, rather than the wire 103 extending all the way through the shaft 106 of the delivery tube and through the collet 107, the wire 103 extends only partially through the shaft 106. A pin 132, which may be rigid or flexible, is positioned within the collet 107 to retain the implant 120. A foreshortening element 133 in the form of flexible filament connects the wire 103 to the pin 132. The foreshortening element 133 may be provided with a length that exceeds the gap between the wire 103 and pin 132, permitting a degree of slack in the foreshortening element 133. This slack may accommodate routing of the delivery tube shaft 106 through a tortuous path without placing the wire 103 into tension or compression. Accordingly, the pin 132 may remain seated in the collet 107 without prematurely releasing the implant 120. Although shown in conjunction with the retention mechanism of FIGS. 3A-3E, it should be appreciated that the actuator of FIG. 17 can be used with any other example of a retention mechanism disclosed herein.

In the description, specific details have been set forth describing some examples. Numerous specific details are set forth in order to provide a thorough understanding of the examples. It will be apparent, however, to one skilled in the art that some examples may be practiced without some or all of these specific details. The specific examples disclosed herein are meant to be illustrative but not limiting. One skilled in the art may realize other elements that, although not specifically described here, are within the scope and the spirit of this disclosure.

Elements described in detail with reference to one example, example, implementation, or application optionally may be included, whenever practical, in other examples, implementations, or applications in which they are not specifically shown or described. For example, if an element is described in detail with reference to one example and is not described with reference to a second example, the element may nevertheless be claimed as included in the second example. Thus, to avoid unnecessary repetition in the foregoing description, one or more elements shown and described in association with one example, implementation, or application may be incorporated into other examples, implementations, or application unless specifically described otherwise, unless the one or more elements would make an example or implementation non-functional, or unless two or more of the elements provide conflicting functions. Similarly, it should be understood that any particular element, including a system component or a method process, is optional and is not considered to be an essential feature of the present disclosure unless expressly stated otherwise.

Any alterations and further modifications to the described devices, systems, methods, and any further application of the principles of the present disclosure are fully contemplated as would normally occur to one skilled in the art to which the disclosure relates. In particular, it is fully contemplated that the features, components, and/or steps described with respect to one example may be combined with the features, components, and/or steps described with respect to other examples of the present disclosure. In addition, dimensions and temporal relationships provided herein are for providing specific examples and it is contemplated that different sizes, dimensions, relationships and/or ratios may be utilized to implement the concepts of the present disclosure. To avoid needless descriptive repetition, one or more components or actions described in accordance with one illustrative example can be used or omitted as applicable from other illustrative examples. For the sake of brevity, the numerous iterations of these combinations will not be described separately. For simplicity, in some instances the same reference numbers are used throughout the drawings to refer to the same or like parts.

The methods described herein are illustrated as a set of operations or processes. Not all the illustrated processes may be performed in all examples of the methods. Additionally, one or more processes that are not expressly illustrated or described may be included before, after, in between, or as part of the example processes. One block element being illustrated separate from another block element does not necessarily require that the functions be performed are illustrated separately.

In some instances, well-known methods, procedures, and components have not been described in detail so as not to unnecessarily obscure aspects of the examples.

While certain exemplary examples of the present disclosure have been described and shown in the accompanying drawings, it is to be understood that such examples are merely illustrative of and not restrictive on the broad disclosure herein, and that the examples of the present disclosure should not be limited to the specific constructions and arrangements shown and described, since various other modifications may occur to those ordinarily skilled in the art.

1. An implant manipulation system, comprising:
a delivery tube;
a collet comprising a plurality of flexible fingers, the collet having an outer diameter in a resting configuration; and
an actuator configured to extend through the delivery tube and the collet and to displace the plurality of fingers radially outward into an expanded configuration, the collet having an outer diameter in the expanded configuration that is greater than the outer diameter in the resting configuration.

2. The implant manipulation system of claim 1, wherein the actuator comprises a wire and the plurality of flexible fingers is configured to return to the resting configuration upon removal of the wire from the collet.

3. The implant manipulation system of claim 2, wherein the actuator further comprises a handle at a proximal end of the wire, the handle configured to slide relative to a hollow base portion of the delivery tube.

4. (canceled)

5. The implant manipulation system of claim 1, wherein the collet is partially disposed within a working channel of the delivery tube.

6. (canceled)

7. The implant manipulation system of claim 1, wherein at least one flexible finger of the plurality of flexible fingers comprises a flange extending radially outward from a distal end of the at least one flexible finger.

8. The implant manipulation system of claim 1, wherein the collet comprises a central channel extending from a proximal end of the collet through the plurality of flexible fingers.

9. The implant manipulation system of claim 1, wherein a first flexible finger of the plurality of flexible fingers has a length that exceeds a length of a second flexible finger of the plurality of flexible fingers.

10. The implant manipulation system of claim 1, wherein an outer surface formed by the plurality of flexible fingers is threaded.

11. The implant manipulation system of claim 1, further comprising a retainer, wherein the retainer comprises a tubular member with a cavity and an internal lip disposed at a proximal end of the cavity.

12. The implant manipulation system of claim 11, wherein the retainer is configured to be affixed to a proximal end of an implant.

13. The implant manipulation system of claim 11, wherein a diameter of an opening formed by the internal lip is less than the outer diameter of the collet in the expanded configuration and greater than the outer diameter of the collet in the resting configuration.

14. The implant manipulation system of claim 11, wherein the retainer is integrally formed with an implant.

15. The implant manipulation system of claim 14, wherein the implant comprises an embolic coil or a stent.

16. The implant manipulation system of claim 1, further comprising a retainer secured to a distal end of the delivery tube, wherein the retainer comprises a tubular member with a cavity and an internal lip disposed at a distal end of the cavity.

17. The implant manipulation system of claim 16, wherein the collet is configured to be secured to a proximal end of an implant and a diameter of an opening formed by the internal lip is less than the outer diameter of the collet in the expanded configuration and greater than the outer diameter of the collet in the resting configuration.

18. The implant manipulation system of claim 1, further comprising a retainer secured to a distal end of the delivery tube, the retainer comprising a central channel configured receive legs of a stent when the stent is in a collapsed configuration, wherein the outer diameter of the collet in the resting configuration is less than an inner diameter of the stent in the collapsed configuration and the outer diameter of the collet in the expanded configuration is greater than the inner diameter of the stent in the collapsed configuration.

19. (canceled)

20. The implant manipulation system of claim 1, wherein the collet is configured to slide longitudinally with respect to the delivery tube.

21. The implant manipulation system of claim 20, wherein the collet comprises a flange positioned within a chamber in the delivery tube, the flange having an outer diameter that is greater than an inner diameter of the delivery tube proximal of the chamber and distal of the chamber.

22-30. (canceled)

31. An implant system, comprising:
a delivery tube with a collet secured to a distal end of the delivery tube;
a flexible coil implant; and
a filament having a distal end secured to a distal end of the implant and having a free proximal end, the filament configured to be gripped between the collet and an internal wall of the implant when the implant is secured to the delivery tube by the collet.

32-35. (canceled)

36. An implant, comprising:
a stent or an embolic coil; and
a collet secured to a proximal end of the stent or embolic coil and having a plurality of flexible fingers, wherein the collet is configured for receipt within a retainer secured to a delivery tube and is configured to receive a portion of an actuator between the plurality of flexible fingers to expand the collet into an expanded configuration in which the collet is interlocked with the retainer.
